(19) United States
(12) Patent Application Publication
(10) Pub. No.: US 2023/0293677 A1
(43) Pub. Date: Sep. 21, 2023

(54) USE OF A DERIVATIVE OF SYNTHETIC PHTHALHYDRAZIDE DERIVATIVES

(71) Applicants: Musa Tazhudinovich ABIDOV, Moscow (RU); Naki KARAASIAN, Moscow (RU)

(72) Inventors: Musa Tazhudinovich ABIDOV, Moscow (RU); Naki KARAASIAN, Moscow (RU)

(21) Appl. No.: 18/016,650

(22) PCT Filed: Jun. 29, 2021
(86) PCT No.: PCT/RU2021/000277

§ 371 Date: Jan. 17, 2023

(30) Foreign Application Priority Data
Jul. 17, 2020 (RU) 2020123853

(51) Int. Cl. A61K 39/39; A61K 39/12; A61K 31/502; A61P 37/04; A61P 31/00

(52) U.S. Cl. CPC A61K 39/39; A61K 39/12; A61K 31/502; A61P 37/04; A61P 31/00; A61K 2039/545

(57) ABSTRACT

The invention relates to the microbiology, virology and immunology. It is intended to form the body immunity to infections of various nature and increase the specific activity of immunobiological drugs. An increase in the body's immunity to infections of various nature and the specific activity of immunobiological medicinal products (IMP) is ensured by the administration of phthalhydrazide derivative (hereinafter referred to as Abidov's adjuvant) in a single dose of 150 μg/individual as an adjuvant (IMP) to animals. The use of the invention makes it possible to increase the effectiveness of specific infection prevention regardless of the IMP nature, and, thereby, increasing efforts against actual controllable infectious diseases of various etiologies. 2 independent and 1 dependent claims, 2 illustrations, 6 tables, 6 examples

The invention relates to immunology, virology, biotechnology and is aimed at increasing the specific activity of immunobiological medicinal products (IMP), can be used for specific immunological prophylaxis of controlled infections of various etiologies.

The invention is implemented by using a phthalhydrazide derivative, which is an agent with immunotropic and anti-inflammatory properties as adjuvant. Anti-inflammatory agents belong to drugs that regulate functional and metabolic activity of macrophages and neutrophilic granulocytes, and have a pronounced anti-inflammatory effect.

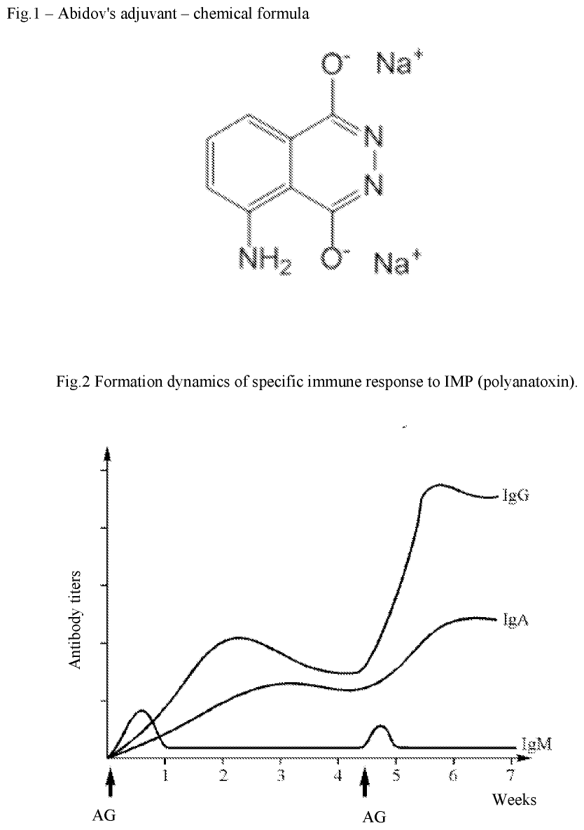

The formula of Abidov's adjuvant is a derivative of synthetic phthalhydrazide derivatives; other derivatives of phthalhydrazide, having a similar formula, obtained by synthetic or other methods in the form of dihydrate, monohydrate, anhydrate and with any other salts (FIG. 1).

Abidov's adjuvant—chemical formula:

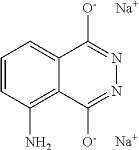 [SMILES: Nc1cccc2c([O-])nnc([O-])c12 ; [Na+] ; [Na+]]

Basic mechanisms of immunotropic action:

nonspecific immunological resistance system—regulates the macrophage activity, regulates the production of reactive oxygen species and other acute phase proteins responsible for the toxic syndrome development; normalizes the functional state of macrophages, leads to the restoration of their antigen-presenting and regulating functions, thereby enhancing the processes of specific antibody formation by at least 3-4 times; enhances the functional antibacterial activity of neutrophilic granulocytes, complete phagocytosis, and increases the nonspecific immunological resistance of the body;

cytokine system—reduces excessive synthesis of TNF, interleukin (IL)-1; enhances the interaction between T- and B-cells and, thereby enhances specific immune response, increases antigen-presenting activity of cells.

Infectious diseases of various nature continue to be a significant problem of modern infectology and medicine. Much importance is attached in this aspect to dangerous and especially dangerous infections (DEDI), which include plague, anthrax, tularemia, viral hemorrhagic fevers, encephalitis, etc. [1, 2]. Outbreaks of these infections are recorded primarily in developing countries with unstable socioeconomic conditions according to available publications [3-6]. These infections have also not lost their relevance today for developed countries, including Russia, as evidenced by:
periodic activation on the country territory of natural foci of anthrax, hemorrhagic fever with renal syndrome, Crimean-Congo hemorrhagic fever, tick-borne encephalitis, etc.;
periodic outbreaks of West Nile fever, bird flu, Californian encephalitis, Japanese encephalitis, Sakhalin fever, Issyk-Kul fever and other dangerous infections [7].

Analysis of the current sanitary and epidemiological situation shows that an epidemiologically unfavorable situation persists for a number of bacterial and viral infections in some countries adjacent to the Russian Federation. In addition, at present, the unauthorized use of infectious agents, including DEDI, for the implementation of bio-terrorist attacks is not excluded, Priority in such conditions and for the implementation of such goals will be given to non-traditional genetically infectious agents, against which the existing means of specific immunological prophylaxis may not be effective enough according to domestic and foreign experts [7].

Researches on the search for effective means of protection against infections, especially those that suddenly appear in the human population and immediately cause panic, chaos, socio-economic cataclysms, etc., seem relevant in view of the aforesaid. The latter include zoonotic transmissible avian influenza viruses, swine influenza viruses, coronaviruses that cause SARS, MERS, SARS-CoV-2, COVID-19, etc., which can infect large population groups and cause epidemics in the shortest possible time. The main danger of these bioagents is not in the mass character of the lesions caused by them, but in the absence of adequate countermeasures due to their sudden appearance. Moreover, the available remedies, in particular, antibacterial and antiviral drugs are not always effective.

It is commonly known that the body resistance to pathogens is the ability of the body to endure the effects of pathogens and form immune response to them. A viable organism is able to return to the health state after a response to infection [8-10]. Most currently available treatments for infectious diseases are antibacterial drugs and thus target the pathogen itself. Increased attention to rapid clearance of pathogens seems to be the highest priority given the damage that they can cause to the body. At the same time, in case of infection with various pathogens, an equally important medical intervention is increasing the ability of the macroorganism to adequately tolerate the direct and/or indirect effects of the pathogen [11-14]. In a certain aspect, it is possible in case of preliminary early cycle of specific immunological prophylaxis using IMP.

IMP is one of the most effective and widely used means of antiinfective protection. They represent an antigenic irritant for a macroorganism specific to a particular infection of bioagent, aimed solely at the formation of its immune defense against a similar but virulent pathogen of the bioagent. The basic criterion allowing the use of IMP in clinical practice is harmlessness: IMP should not have side effects and cause the development and progression of the corresponding infectious disease in vaccinated people. At the same time, it should be noted that the main criterion characterizing IMP in terms of its practical application is efficiency. The mentioned criterion indicates that IMP should ensure the development of intense immunity to a specific infection and its maintenance for a long (at least 12 months) period of time after vaccination [15-19].

The composition of modern IMP includes actually a specific antigen (immunogen) and excipients that do not have a negative effect on both the immunogen itself and the vaccinated body. Some IMP may include antigen—the active principle, substances that activate the antigen action—adjuvants, as well as stabilizers and preservatives. Obviously, the effectiveness of IMP is determined primarily by the properties of the immunogen included in its composition. Available IMPs differ significantly in the nature of used immunogen [16, 20]. The most widely used IMP in infectology are preparations on the basis of live attenuated strains of the relevant microorganisms (for example, vaccines against anthrax, plague, tularemia, yellow fever, etc.) and preparations on the basis of inactivated killed strains of microorganisms, as well as the chemical structures of individual microorganisms, recombinant structures, DNA vaccines, etc.

IMP on the basis of attenuated strains of microorganisms are characterized by the presence in their composition of whole strains of microorganisms that have not lost their antigenic properties, but are devoid of virulence. As a rule, these IMPs are highly effective, but they require constant monitoring due to the vaccine strain reversion and the acquisition of virulent properties. Attenuated vaccines can be used not only for immunization against a specific pathogen, but also against a heterologous infection due to a certain similarity between pathogens (for example, plague vaccines can be used for specific immunological prophylaxis of tularemia due to the common antigenic structure of pathogens).

IMPs constructed on the basis of killed strains of pathogens, their antigenic determinants, genetic sequences are more preferable in terms of safety in comparison with live attenuated vaccines, that is, they are less reactogenic. However, these drugs are inferior to them in terms of effectiveness. Moreover, the duration of specific immunity formed during their use is approximately 1.5-2 times shorter than during immunization with live vaccines. Currently, almost all IMPs approved for use in the territory of the Russian Federation are included in the National calendar of preventive vaccinations.

Thanks to genetic sequence decoding of many pathogens, it is currently possible to obtain IMP of known specificity and structure [21, 22]. Attenuated (weakened) strains of high efficiency and safe for humans are purposefully constructed by changing the genotype of pathogens by deletions or point mutations of virulence genes. In particular, influenza vaccines have been developed and continue to be developed by this way [20]. Cloning of protective antigens genes of various pathogens has opened the possibility of obtaining recombinant IMP producers. These include recombinant strains of viruses, bacteria, yeast, providing the expression of these genes and, accordingly, the synthesis of antigens of known structure. The most convenient for use as a vector were vaccinia virus, adenoviruses, vesicular stomatitis virus, etc. Vaccines against hepatitis B (Revax-B), Ebola hemorrhagic fever and other infections were developed with their help, or rather on their platform. In addition, it is possible when cultivating recombinant producer strains to obtain sufficient amounts of certain protective antigens, on the basis of which molecular vaccines can be further designed.

In some cases, idiotypic vaccines are used for immunization due to the high toxicity of the antigen, containing anti-idiotypic antibodies as an active principle that reproduce the antigen configuration. The effective vaccine against the hepatitis B virus has been designed based on this principle.

Another way to create specific vaccines of known structure is based on the use of chemically synthesized peptides corresponding to certain antigen epitopes. These peptides are usually non-immunogenic, that is why they are conjugated with carriers in the form of a protein or a high molecular weight polymer. Synthetic vaccines of this type are created against salmonellosis and influenza. The emergence and development of gene therapy has opened up a qualitatively new way of immunological prophylaxis based on the creation of DNA vaccines. Genetic engineering methods can be used to obtain any genetic constructs that include protective antigen gene. Plasmids are used as a vector, as well as a number of viruses to transfer and express cytokine genes. When the vector is injected intramuscularly into myocytes, the specified gene expression is occurred, which ensures the development of a specific immune response. Safety of the method and the possibility of targeted immunological prophylaxis open up great prospects for the use of DNA vaccines [23]. The so-called “therapeutic vaccines” are also should be mentioned. This notion is used to designate bacterial vaccines and toxoids that induce not only protective immunity, but also cause pronounced immunomodulation [24, 25]. Vaccine therapy has found wide application in the treatment of cancer patients, elimination of chronic disease recurrences, as well as in the relief of allergic diseases.

The strength of the immune response depends on two main factors: macroorganism properties and characteristics of the antigens used for immunization. The immunogenicity of antigens obtained from pathogens of infectious diseases is not the same. Exotoxins and surface antigens of microorganisms are the most immunogenic. Vaccine immunogenicity largely depends on how well the antigens are chosen for designing the drug. Nonspecific immunostimulants (adjuvants) are used with insufficient immunogenicity. Aluminum hydroxide, aluminum phosphate, calcium phosphate, polyoxidonium and protein carriers are used as immunostimulants in the vaccination practice. An example is the Grippol IMP, which contains polyoxidonium as an adjuvant. Difficulties in creating highly effective vaccines are associated with macroorganism characteristics, its genotype, phenotype, with the existence of two types of immunity (humoral and cellular), which are regulated by different subpopulations of helper cells (Th1 and Th2). Post-vaccination immunity consists of two types of immune reactions: humoral and cellular. The absence of circulating antibodies is not yet evidence of a weak immune system; immune response develops upon a new encounter with an antigen due to immunological memory. In addition, resistance to certain types of infections is based on cellular mechanisms, therefore vaccines used to prevent these infections should form cellular immunity.

IMP immunogenicity is the basis of its effectiveness [17, 18]. As a rule, IMP corpuscularity (live, killed) provides the necessary immunogenicity, in other cases additional methods to increase IMP immunogenicity should be often used. The main ways to increase the IMP immunogenicity include: the use of optimal antigen concentration; vaccine purification from low molecular weight substances that can cause specific or nonspecific suppression of the immune response; antigen aggregation using covalent binding and other complexation methods; inclusion in the vaccine of the maximum amount of antigen epitopes; sorption on substances that create an antigen depot (aluminum hydroxide, calcium phosphate, etc.); the use of liposomes (water-oil emulsion); adding microbial, plant, synthetic and other types of adjuvants; binding of weak antigen to protein carrier (tetanus, diphtheria toxoid, etc.); inclusion of the antigen in microcapsules ensuring antigen release after a specified period of time; improvement of conditions for processing and presentation of the antigen. Use of class I and II histocompatibility antigens or antibodies to these antigens. Approaches to IMP creation that ensure the formation of cellular and humoral immunity are different. This is due to the participation of two regulatory cells in the immune response: Th1 and Th2. There is a certain degree of antagonism between them, although they are formed from one kind of progenitor cells. It is quite difficult to obtain IMP that would induce cellular immunity. In many cases, it is not possible to switch the Th2 immune response to a vaccine that stimulates the antibody production to a Th1 cellular response. It is essential that IMP induces T-dependent immune response. Otherwise, the response will be short-term, and repeated administration of the drug will not cause a secondary response. Primary and secondary immune response differ from each other in the immunity formation dynamics (FIG. 2). The secondary immune response is not sufficiently pronounced if a weakly immunogenic antigen is used for immunization, if passively administered or actively acquired antibodies are present in the body, or if the antigen is administered to a patient with immunodeficiency. The secondary immune response is characterized by the following features: earlier (compared to the primary response) development of immune reactions; reducing the antigen dose required to achieve an optimal response; increase in the strength and duration of the immune response; enhancing humoral immunity by: increasing the number of antibody-forming cells and circulating antibodies; activation of Th2 and increasing the production of cytokines (IL-3, -4, -5, -6, -9, -10, -13, GM-CSF, etc.); shortening the period of IgM-antibody formation, predominance of IgG- and IgA-antibodies; increase in antibody affinity; strengthening of cellular immunity by: increasing the number of antigen-specific T-killers and delayed-type hypersensitivity T-effectors; activation of Th1 and increased production of cytokines (IF-γ, FIO, IL-2, GM-CSF, etc.); increasing the affinity of antigen-specific T-cell receptors; increasing resistance to infection.

The body acquires the ability to quickly respond to repeated contact with an antigen due to immunological memory. It is characteristic of cellular and humoral immunity and depends on the formation of T- and B-memory cells. Immunological memory develops after previous infection or vaccination and persists for a long time. Antibodies after some infections have been present in blood serum for decades. However, the half-life of the most stable immunoglobulin averages 25 days. Thus, the resynthesis of specific immunoglobulin is constantly taking place in the body. The duration of post-infectious immunity depends on the pathogen properties, infectious dose, immune system state, genotype, age, and other factors. Immunity can be short-term, for example, after influenza, dysentery, relapsing fever; long enough, for example, after anthrax, rickettsiosis, leptospirosis; and even lifelong, for example, after polio, measles, whooping cough. Acquired immunity is a good defense against infection by the relevant pathogen. If the main mechanism of immunity after a given infection is the neutralization effect, then the presence of a certain level of circulating antibodies is sufficient to prevent reinfection. The vaccine has to be administered 2 times or more to achieve stable immunity. Primary vaccination can consist of several doses of the vaccine, the intervals between doses are strictly regulated. The revaccination schedule is more free, revaccination can be carried out in a year and even in several years. The interval between vaccine administrations should be at least 4 weeks. Otherwise, less stable immunity develops. Conversely, some increase in the 4-week interval can enhance the secondary immune response. The maximum increase in the antibody concentration in the secondary response to vaccines occurs at low initial antibody titers. High pre-existing level of antibodies prevents additional production of antibodies and their long-term preservation, and a decrease in antibody titers is observed in some cases [26-40].

However, traditionally used adjuvants are also have drawbacks in terms of enhancing IMP immunogenicity. Thus, adjuvants of bacterial origin (for example, Freund's adjuvant), after administration to the body contribute to the development of a large number of adverse reactions. Aluminum compounds, despite their widespread use, sharply increase allergenic properties of IMP, in addition, they are effective only against those antigens that require humoral immunity induction.

Currently, drugs with selective targeted action are preferred when searching effective adjuvants, and this search has expanded significantly due to the progress made towards decoding immunoreactivity mechanisms. Researches have shown that all its links are under the control of cytokines. The latter are synthesized by various types of cells involved in the development of immune response and are characterized by a certain action specificity [40-42]. Recent achievements in immunopharmacology allow a new approach to this problem. In particular, it is known that, when used together with vaccine preparations, adjuvants have different effects on the components and immune response stages (macrophages, B-, T-cells and their subpopulations, natural killers, migration and cooperation processes, etc.). This creates the fundamental possibility of differentiated use of adjuvants, focused on specific target cells. Selective modulation of certain immunity links by adjuvants when they are administered with vaccines to increase immunogenicity of the latter may turn out to be a very promising direction in vaccinology. For this purpose, drugs that have a regulating effect on the functional state of the monocyte-macrophage link cells of the immune system can be used, ensuring in this regard the balance, specificity and effectiveness of the body's immune responses to antigenic effects.

Taking into account the abovementioned, it can be concluded that some immunomodulatory agents can be used at present as adjuvants for IMP. At the same time, these drugs mainly have an immunomodulatory effect, activating processes of specific antibody genesis in relation to the IMP used in combination, but at the same time triggering both immunogenesis and a pronounced inflammatory reaction, without which a full-fledged immune response is not possible according to researchers. However, an increased inflammatory reaction can also lead to undesirable consequences, namely, reactogenicity of vaccinations, formation of immunodeficiency in the post-vaccination period, etc. In this regard, the task of searching for vaccine adjuvants, which, on the one hand, contributed to IMP immunogenicity increase, and, on the other hand, neutralize its side effects, in particular, those associated with post-vaccination inflammation, remains relevant. Thus, the use of synthetic derivatives of phthalhydrazide derivatives as adjuvants, in particular, Abidov's adjuvant, is not obvious to a specialist. Such substances are known to be capable of regulating the activity of macrophages at the level of nonspecific immunological resistance, respectively, regulating the production of reactive oxygen species and other acute phase proteins responsible for the toxic syndrome development. They increase the functional antibacterial activity of neutrophilic granulocytes, enhance phagocytosis and increase the nonspecific defense of the body. It reduces the excessive synthesis of TNF and IL-1 at the level of the cytokine system. Both T- and B-lymphocytes are activated at the level of T- and B-systems of immunological defence. It enhances the immune response after various infections (Abidov M. T. Toxic syndrome, pathogenesis, correction methods, 1994, Bulletin of experimental biology and medicine, 2000). Taking into account the abovementioned, it can be assumed that phthalhydrazide derivatives can be used as IMP adjuvants (hereinafter referred to as Abidov's adjuvant) in order not only to increase their immunogenicity, but also, which is especially important, to reduce the side effects of IMP, which is not mentioned in the available literature.

Compounds are known among these substances that have a certain anti-infective activity [43], in particular, furunculosis of staphylococcal and streptococcal etiology, herpetic infection, and Mycobacterium tuberculosis [44, 45]. Some phthalhydrazide derivatives are used in the treatment of pancreatic necrosis [46], bronchial asthma [47], periodontitis [48, 49], and viral infection [50].

The technical results of the claimed invention are:
manifestation of adjuvant effect, i.e. increasing immune response, with a joint, separate (in different anatomical areas of the body) administration of Abidov's adjuvant with IMP, which is unexpected in the context of state of the art. A specialist could not expect both the manifestation of the adjuvant effect itself, as well as, moreover, its manifestation precisely with simultaneous (simultaneous administration is the minimum possible interval between the administration of Abidov's adjuvant and the immunogen, antigen) separate administration with IMP;
increasing IMP immunogenicity, and on the other hand, leveling the side effects of IMP, in particular, those associated with post-vaccination inflammation;
reducing reactogenicity of vaccinations, reducing cases of immunodeficiency formation in the post-vaccination period;
increasing immunogenicity of inactivated and chemical vaccines at least 3 times;
increasing safety of live vaccines, without affecting the immunogenicity, by means of reducing the reactogenicity;
increasing immunogenicity of polyanatoxin associations by at least 30% of each antigen (toxoid) included in the association;
activation of regenerative processes at the tissue level in case of damage by virus, for example, lungs. It was impossible to assume the effect of Abidov's adjuvant on the regeneration of, for example, lungs, from the state of art.

The purpose of the invention is to increase the effectiveness of the fight against infectious diseases caused by pathogens of dangerous and especially dangerous infections of various etiologies, as well as the immunological effectiveness of immunobiological drugs used for this.

The purpose is achieved in that a phthalhydrazide derivative, Abidov's adjuvant, is used in a single dose of 150 μg/individual and, or in a different dose, from 0.01 to 1000 μg/individual, depending on the subject in need, administered once or multiple times, intramuscularly, together and/or separately from the appropriate immunobiological drug for a particular infection in order to increase the effectiveness of immunological prophylaxis of dangerous and especially dangerous infections of various etiologies.

Table 1 presents an assessment of the adjuvant activity of Abidov's adjuvant in relation to IMP against herpetic infection.

Table 2 presents an assessment of the adjuvant activity of Abidov's adjuvant in relation to IMP against Venezuelan equine encephalomyelitis.

Table 3 presents an assessment of the adjuvant activity of Abidov's adjuvant in relation to IMP against orthopoxvirus infections.

Table 4 presents an assessment of the adjuvant activity of Abidov's adjuvant in relation to IMP against botulism and tetanus.

Table 5 presents an assessment of the adjuvant activity of Abidov's adjuvant in relation to IMP against typhoid fever and dysentery.

Table 6 presents an assessment of the adjuvant activity of Abidov's adjuvant in a comparative aspect in relation to IMP against cowpox and Venezuelan equine encephalomyelitis.

The method is implemented using Abidov's adjuvant in a single dose of 150 μg/individual intramuscularly simultaneously or separately from IMP to increase IMP immunogenicity.

Abidov's adjuvant is the sodium salt of 5-amino-1,2,3,4-tetrahydrophthalazine-1,4-dione, the sodium salt, an immunostimulating agent that has an adjuvant action. It affects the levels of pro-inflammatory cytokines responsible for the toxic syndrome development. It also reduces the levels of reactive oxygen radical anions responsible for the fibrosis formation in the lungs. It normalizes the functional state of macrophages, leads to the restoration of their antigen-presenting and regulating functions. It increases the functional antibacterial activity of neutrophilic granulocytes, enhance phagocytosis and increase the nonspecific defense of the body. The drug can be used for acute and chronic diseases of various origins accompanied by intoxication, as well as for purulent-septic complications—as part of complex therapy.

There are specific examples of method implementation below.

### Example 1. Adjuvant Properties of Abidov's Adjuvant in Relation to IMP Against Herpetic Infection

Herpes simplex virus (HSV) of type 1, US strain, initial virus titer 10²-10³ LD₅₀/ml or HSV of type 2, strain VN, initial virus titer 10²-10³ LD₅₀/ml were used to model herpetic infection. The studies were performed on outbred mice obtained from the Rappolovo nursery of the Russian Academy of Sciences. 300 outbred male mice in total weighing 16-18 g were used in the studies. The experiments were carried out on animals that were obligatorily quarantined for 1 week. Suckling mice were used for intracerebral accumulation of the model virus, as well as to determine the infectious activity of the virus-containing material. HSV accumulation was carried out by intracerebral infection of suckling mice. Infectious activity of virus-containing materials was determined by the Reed-Mench method. LD₅₀ value for each pathogen was determined on outbred mice using the Kerber method modified by I. P. Ashmarin and A. A. Vorobyov (1962). Infection was carried out by subcutaneous infection of animals with a suspension of herpes simplex virus of type 1 in a volume of 0.5 ml/individual at a dose of 2.5 LD₅₀. To increase the susceptibility of animals to infection, they were subjected to a single irradiation 4 days before infection at the IGUR-1 unit at a dose rate of 1.5 Gy/min. The cumulative dose of radiation was 5.5 Gy. Each experimental and control group numbered 10 individuals. The infection was carried out 28 days after the immunization of the animals. Herpetic cultural inactivated dry vaccine (HV) was used for immunization (ser. 126, control No. 718). The immunizing dose of IMP was 0.1 of human dose. The vaccine was administered once subcutaneously in a volume of 0.5 ml at the withers of the animals. Abidov's adjuvant was administered to animals intramuscularly in the right thigh of the hind paw at a dose of 150 μg/individual. Two immunization schemes were tested:
Abidov's adjuvant was administered simultaneously—separately from IMP;
Abidov's adjuvant was administered against the background of the current immunization process—3, 2, 1 days before infection.

Animals of the control groups were administered: only IMP, only Abidov's adjuvant, only saline sodium chloride solution.

Results obtained are reported in the Table 1.

TABLE 1
Assessment of the adjuvant activity of Abidov's
adjuvant in relation to IMP against herpetic infection.

| | | Number of | Number of | | Number of | |
|---|---|---|---|---|---|---|
| | Infectious dose | animals in | dead | | survived | |
| | of the pathogen | the group, | animals, | | animals, | |
| Drug | (q-ty of _{LD}50) | heads | % | | % | |
| Abidov's adju- | 2.5 | 10 | 20 | (2-56) | 80 | (44-98)* |
| vant + vaccine | | | | | | |
| simultaneously | | | | | | |
| in different places | | | | | | |
| Vaccine + | 2.5 | 10 | 20 | (2-56) | 80 | (44-98) |
| Adjuvant 3, 2, 1 | | | | | | |
| days before | | | | | | |
| immunization | | | | | | |
| Adjuvant | 2.5 | 10 | 80 | (44-98) | 20 | (2-56) |
| single dose | | | | | | |
| Adjuvant 3, 2, 1 | 2.5 | 10 | 70 | (19-81) | 30 | (7-65) |
| days before | | | | | | |
| Vaccine | 2.5 | 10 | 50 | (19-81) | 50 | (19-81) |
| Infection control | 2 | 10 | 100 | (69-100) | 0 | (0-31) |

The results obtained indicate that Abidov's adjuvant has an adjuvant effect on herpesvirus vaccine when used according to all the studied schemes. The survival rate in these groups was 80% that significantly exceeded the same indicator in the infection control (p≤0.05). Compared with immunization with IMP alone or with adjuvant alone, the survival rate in groups of animals that received both IMP and Abidov's adjuvant was 30-60% higher. In case of using adjuvant in immunization schemes, the effectiveness of IMP increased by 30%, which indicates the presence of adjuvant activity in Abidov's adjuvant against the herpetic cultural inactivated dry vaccine. There is no such information in the available sources. The new function of Abidov's adjuvant does not clearly follow from its known properties and composition. The activity of the drug in experiments in vivo against pathogens that cause furunculosis, acute and chronic infections of the gastrointestinal tract, generalized herpetic infection without the use of IMP does not mean that the drug will necessarily have adjuvant properties in relation to the herpetic vaccine, as well as in relation to other IMP.

See the following examples that prove the compliance of the proposed method with the invention criterion “industrial applicability”. Virulent strains of pathogens were used to modeling infections: Venezuelan equine encephalomyelitis (VEE), cowpox (CP), botulism, tetanus, typhoid fever (TF), and dysentery. The studies were performed on outbred male mice weighing 16-18 g. Animals were taken into the study after obligatorily quarantine for 1 week in vivarium conditions. Infectious activity value of pathogens (LD₅₀) was assessed by the Kerber method modified by I. P. Ashmarin and A. A. Vorobyov [51].

The effectiveness of the corresponding IMP when used simultaneously with Abidov's adjuvant and without it was determined by comparison of the survival rates of animals in experimental (IMP immunized animals with Abidov's adjuvant) and control groups. The percentage of survived animals in the experimental and control groups was determined according to the tables of Genes V. S. [52]. Observation of infected animals was carried out for 21 days with daily record of living and dead individuals in the experimental and control groups. In addition, the protective efficiency (%) of Abidov's adjuvant was determined, which is the difference between the number of survived infected animals in the experimental group (Abidov's adjuvant was administered, %) and the number of survived infected animals in the control group (Abidov's adjuvant was not administered, %), as well as the average life expectancy T, (ALE, days) of experimental and control animals. The latter was calculated by the formula:

$$T = \frac{\sum ( N \times S )}{N 1} \qquad ( 1 )$$

where N—number of survived animals, heads;
   S—observation period, days;
   Ni—number of infected mice in the group, heads
Statistical analysis of the study results was carried out using the computer program for statistical data processing Statistica 6.0 for Windows. To assess quantitative indicators, standard quantitative characteristics were determined: average value of the indicator (M), standard deviation, standard error of the mean value (m). Comparison of quantitative data was performed using paired and unpaired Student's test using Student's t-test. The results are presented as M±m. Differences were considered significant at p≤0.05.

### Example 2. Adjuvant Effect of Abidov's Adjuvant on IMP Against Venezuelan Equine Encephalomyelitis

The evaluation of the adjuvant properties of Abidov's adjuvant in relation to the vaccine of Venezuelan equine encephalomyelitis (VVE) was carried out in “point” experiments, 10 mice weighing from 16 to 18 g were used at each point. Virus-containing material was administered to animals subcutaneously in a volume of 0.3 ml/mouse. The infectious doses of the virus were 2 and 10 of LD₅₀. The virus of Venezuelan equine encephalomyelitis (VEL), strain Trinidad was used for infection. The initial virus titer was 10⁷-10⁸ LD₅₀/ml. Cultural inactivated liquid VVE (VVE) was used for immunization (ser. 145; control No. 1244). The adjuvant was used at a dose of 150 μg/individual, administered intramuscularly according to various schemes. IMP was used once, administered intramuscularly in a volume of 0.5 ml, immunizing dose of IMP was 0.1 of human dose.

As follows from the presented data, VVE ensured the survival rate of 15% (10 LD₅₀) and 70% (2 LD₅₀) of infected animals administered once at a dose equal to 0.1 of human dose 21 days before infection, depending on the infecting dose of the virus.

The results of conducted studies are presented in Table 2.

TABLE 2
Assessment of the adjuvant activity of Abidov's adjuvant in relation
to IMP against Venezuelan equine encephalomyelitis

| | | Infectious | | |
|---|---|---|---|---|
| Scheme of Abidov's | | dose of the | | |
| adjuvant administration | Method | pathogen | Survival rate, | |
| in relation to | of adjuvant | (LD₅₀ | M (M − tm − | |
| VVE and infection | administration | amount) | M + tm), % | |
| Simultaneously with VVE | Intramuscularly | 10.0 | 15 | (2-45) |
| in one syringe 21 days | | 2.0 | 40 | (12-74) |
| before infection | | | | |
| Simultaneously with VVE | Intramuscularly | 10.0 | 40 | (12-74) |
| in different syringes 21 days | | 2.0 | 100 | (69-100)* |
| before infection | | | | |
| Separately from VVE 3, 2, | Intramuscularly | 10.0 | 40 | (12-74) |
| 1 days before infection | | 2.0 | 100 | (69-100)* |
| Without VVE 21 days | Intramuscularly | 10.0 | 40 | (12-74) |
| before infection | | 2.0 | 70 | (35-93)* |
| Without VVE 3, 2, 1 days | Intramuscularly | 10.0 | 60 | (26-83) |
| before infection | | 2.0 | 100 | (69-100)* |
| Not administered (control 1) | — | 10.0 | 15 | (2-45) |
| | | 2.0 | 70 | (35-93)* |
| Not administered (control 2) | — | 10.0 | 0 | (0-31) |
| | | 2.0 | 15 | (2-45) |

Control 1 - animals were immunized only with VVE;
Control 2 - VVE and Abidov's adjuvant were not administered;
*differences with indicators in control groups are considered to be significant at P < 0.05

Administration of only Abidov's adjuvant in the same period ensured the survival rate of 40% (10 LD₅₀) and 70% (2 LD₅₀) of mice infected with the VVE virus. The use of Abidov's adjuvant on a multiple scheme was more effective. At the same time, the survival rate of infected mice was 60-100% depending on the infecting dose of the virus.

With simultaneous administration of VVE and Abidov's adjuvant in one syringe, the survival rates of infected animals ranged from 15 to 40% and were either at the level or slightly lower than in groups of animals that were administered with only VVE or only adjuvant. The simultaneous use of VVE and adjuvant in different syringes was more effective. In this case, the survival rate of infected animals was 40% and 100% depending on the size of the infecting dose of the VVE virus, when infected with the virus at a dose of 10 LD₅₀ and 2 LD₅₀ respectively, which turned out to be 25-30% higher than the similar indicator registered in groups of animals, immunized only with VVE.

### Example 3. Adjuvant Effect of Abidov's Adjuvant on IMP Against Orthopoxvirus Infection

The studies were carried out on outbred male mice weighing from 10 to 12 g. Size of groups—20 animals each. Smallpox live dry vaccine (SLDV) was used for immunization (ser. 80, control No. 1841). IMP was administered to animals at a dose of 0.2 of human dose, immunization was carried out once subcutaneously, the volume of the administered vaccine was 0.5 ml. Abidov's adjuvant was used at a dose of 150 μg/individual, administered subcutaneously according to various schemes. Mice were infected intranasally 14 days after immunization. Cowpox virus (CP), Pumenok strain, was used for this. The initial virus titer was 10³-10⁴ LD₅₀/ml. The virus-containing suspension was administered in a volume of 0.1 ml. The infectious doses of the virus were 1 and 10 of LD₅₀. The results of studies are presented in Table 3.

It is established that, regardless of the combined use schemes it was not possible to identify a pronounced adjuvant effect of Abidov's adjuvant in relation to SLDV. The values of survival rates of infected animals, which were injected with Abidov's adjuvant and SLDV, practically did not differ from those recorded in groups of mice immunized only with SLDV. Probable reason for this is that it is quite difficult to obtain an adjuvant effect against such a strong antigen as SLDV. At the same time, no negative effect of the adjuvant on the formation of specific orthopoxvirus immunity was determined when used together with the vaccine. Therefore, this variant cannot be excluded during specific immunological prophylaxis of the above-mentioned disease with the use of SLDV.

TABLE 3
Assessment of the adjuvant activity of Abidov's adjuvant
in relation to IMP against orthopoxvirus infections

| | Infectious | Survival | |
|---|---|---|---|
| Scheme of Abidov's | dose of the | rate, M | |
| adjuvant admini- | pathogen | (M − tm − | |
| stration in relation to | (LD₅₀ | M + | |
| SLDV and infection | amount) | tm), % | |
| Simultaneously with | 10.0 | 40 | (19-64)* |
| SLDV in one syringe | 1.0 | 60 | (36-81)* |
| 14 days before infection | | | |
| Simultaneously with | 10.0 | 60 | (36-81)* |
| SLDV in different | 1.0 | 70 | (46-88) |
| syringes 14 days | | | |
| before infection | | | |
| Separately from | 10.0 | 70 | (46-88)* |
| SLDV 3, 2, 1 days | 1.0 | 70 | (46-88)* |
| before infection | | | |
| Without SLDV 14 | 10.0 | 30 | (12-54) |
| days before infection | 1.0 | 90 | (68-99) |
| Without SLDV 3, 2, 1 | 10.0 | 10 | (1-32) |
| days before infection | 1.0 | 90 | (68-99) |
| Not administered | 10.0 | 60 | (36-81) |
| (control 1) | 1.0 | 70 | (46-88) |
| Not administered | 10.0 | 0 | (0-17) |
| (control 2) | 1.0 | 50 | (27-73) |

Control 1 - animals were immunized only with SLDV;
control 2 - SLDV and Abidov's adjuvant were not administered to animals;
*differences are considered to be significant in comparison with control 2 at P < 0.05

### Example 4. Adjuvant Effect of Abidov's Adjuvant on IMP Against Botulism and Tetanus

It is commonly known that tetraanatoxin (TA) includes toxoids against botulism of A, B, E types and tetanus. The anatoxins mentioned in this preparation are sorbed on aluminum oxide hydrate. TA is applied three times to form a specific protection, while the interval between the first and second injections is 28-30 days, and between the second and third—4-6 months. Studies on assessment of adjuvant properties of Abidov's adjuvant in relation to TA were performed on white mice and guinea pigs. The adjuvant dose was 150 μg/individual.

Immunization was carried out twice with an interval of 30 days. Abidov's adjuvant and TA were administered subcutaneously in the same syringe. The volume of the administered mixture of drugs was 0.5 ml. Animal poisoning with botulinum toxin of type A at a dose of 100 LD₅₀ was used as an infection model. Intoxication modeling was carried out 14 days after the second immunization by intraperitoneal administration of the toxin in a volume of 0.5 ml. The results of conducted studies are presented in Table 4.

TABLE 4
Assessment of the adjuvant activity of Abidov's adjuvant
in relation to IMP against botulism and tetanus

| | | | | | |
|---|---|---|---|---|---|
| | Multi- | Number of | Poisoning | Infectious | Survival rate, |
| | plicity of | animals in | time after | dose of the | M (M − |
| Immunization | immuni- | the group, | immuni- | toxin, | tm − M + |
| drug | zation | heads | zation, days | LD₅₀ | tm), % |

White mice

| | | | | | | |
|---|---|---|---|---|---|---|
| TA + Abidov's | 2 | 20 | 14 | 16 | 100 | (83-100)** |
| adjuvant | | | | | | |
| TA | 2 | 20 | 14 | 16 | 70 | (46-88)* |
| Abidov's adjuvant | 2 | 20 | 14 | 16 | 0 | (0-17) |
| Infection control | — | 20 | 14 | 16 | 0 | (0-17) |

Guinea pigs

| | | | | | | |
|---|---|---|---|---|---|---|
| TA + Abidov's | 2 | 20 | 14 | 16 | 100 | (83-100)* |
| adjuvant | | | | | | |
| TA | 2 | 20 | 14 | 16 | 60 | (35-93)* |
| Abidov's adjuvant | 2 | 20 | 14 | 16 | 0 | (0-17) |
| Infection control | — | 20 | 14 | 16 | 0 | (0-17) |

*differences with indicators in the “infection control” group are considered to be significant at p < 0.05.

The use of only Abidov's adjuvant did not significantly affect the resistance of animals to poisoning with botulinum toxin of type A. The survival rate of mice in this group was 0%. Immunization only with TA provided protection for 60-70% of poisoned animals, depending on their species (white mice or guinea pigs), against the background of 100% lethality in control (p<0.05). The most effective was the combined use of TA and Abidov's adjuvant. In this case, it was achieved 100% survival rate of animals against the background of complete lethality in the control (p<0.05). Moreover, this indicator was significantly higher than in the group of animals vaccinated only with TA (p<0.05). Almost completely identical results were obtained on the model of intoxication with tetanus toxin.

### Example 5. Adjuvant Effect of Abidov's Adjuvant on IMP Against Acute Intestinal Infections

The typhoid Vi-polysaccharide liquid vaccine (VIANVAK) was used for immunological prophylaxis of typhoid infection. The drug was administered once subcutaneously in a volume of 0.5 ml at a dose equivalent to 0.02 and 0.2 of human doses. The dysentery lipopolysaccharide vaccine “Shigellvak” from the Sonne strain was used for immunological prophylaxis of dysentery. The drug was administered once subcutaneously in a volume of 0.5 ml at a dose equivalent to 0.2 of human dose.

Characterization of Model Biopathogens.

Typhoid fever pathogen—Breslau strain. The pathogen suspension was prepared as follows: a microbial culture was grown on Muller-Hinton agar at a temperature of 37° C. for 24 hours. Serial dilutions of the grown microbial suspension were prepared at the end of cultivation according to the turbidity standard with a step of 10 (starting from a dilution of 109 to 102 CFU/ml). The resulting suspension was used for further subcutaneous infection of experimental animals.

Dysentery pathogen—Sh. Sonnei II strain. The pathogen suspension was prepared as follows: a microbial culture was grown on Muller-Hinton agar at a temperature of 37° C. for 24 hours. Seeding was also carried out on Endo medium to confirm the purity. Serial dilutions of the grown microbial suspension were prepared at the end of cultivation according to the turbidity standard with a step of 10 (starting from a dilution of 109 to 102 CFU/ml). The resulting suspension was used for further subcutaneous infection of experimental animals. Studies on the assessment of the immunotropic effects of Abidov's adjuvant when used together with IMP were performed on white outbred male mice weighing 16-18 g (600 heads). The experiments were carried out on animals that were obligatorily quarantined for 1 week.

Modeling of typhoid infection in vivo was carried out on outbred mice weighing 16.0-18.0 g by intraperitoneal infection with a suspension of Breslau strain spores in a volume of 0.5 ml. Observation of infected animals was carried out for 14 days with daily record of living and dead individuals in the experimental and control groups. The specificity of animal death was confirmed bacteriologically—by sowing the spleen of dead animals on a special nutrient medium. Modeling of dysentery infection in vivo was carried out on outbred mice weighing 16.0-18.0 g by intraperitoneal infection with a suspension of Sh. Sonnei II strain in a volume of 0.5 ml. Observation of infected animals was carried out for 14 days with daily record of living and dead individuals in the experimental and control groups. The specificity of animal death was confirmed bacteriologically—by sowing the spleen of dead animals on a special nutrient medium.

Antibodies to typhoid antigens in animals immunized with the typhoid Vi-polysaccharide liquid vaccine “VIANVAK” were determined in blood serum on the 21st day after immunization using a reagent kit for detecting antibodies to bacteria antigens of the typhoid-paratyphoid group, brucella and proteus in the agglutination reaction (“Anti-Bactantigen-Test”) (ser. 04/15). The reaction was set up in accordance with the “Instruction . . . ” for use of the kit. Antibodies to Sonne shigellosis antigens were determined in the blood serum of animals immunized with the corresponding IMP on the 21st day after immunization with the drug “Replan”—erythrocyte shigellosis Sonne antigenic diagnostic agent (lyophilisate for diagnostic purposes) (ser. 950914). The diagnostic agent was used according to the “Instructions . . . ” for use. Determination of the titer of anti-shigella antibodies was carried out by TPHA.

Animals were immunized in the course of the studies according to the scheme: animals were initially immunized with the corresponding IMP, then simultaneously with the IMP, and then animals were injected with Abidov's adjuvant intraperitoneally in a volume of 0.5 ml at a single dose of 150 μg/individual 3 days and 6 days after IMP immunization.

Blood was taken from the animals by decapitation before immunization and 21 days after the last administration of adjuvant, and the titers of the corresponding specific antibodies were determined in the blood serum.

The results of conducted studies are summarized in Table 5.

TABLE 5
Assessment of the adjuvant activity of Abidov's adjuvant in
relation to IMP against typhoid fever and dysentery

| | | The values of IMP immunological | |
|---|---|---|---|
| | Quantity | efficiency indicators when it is | |
| | of | determined by serological methods | |
| | immunized | | Reciprocal value of |
| IMP for | animals, | Seroconversions, % | antibody titer, Me |
| immunization | heads | (to O-antigen) | (to O-antigen) |

Typhoid fever (VIANVAK vaccine)

| VIANVAK + | 20 | 100 | (83-100)* | 256 | (128-512*)** |
|---|---|---|---|---|---|
| Abidov's adjuvant | | | | | |
| VIANVAK | 20 | 80 | (56-94)* | 64 | (16-64)* |
| Not administered | 20 | 10 | (1-32) | 8 | (4-16) |

(control)

Dysentery (SHIGELLVAK vaccine)

| SHIGELLVAK + | 20 | 100 | (83-100)*)** | 256 | (128-512)*)** |
|---|---|---|---|---|---|
| Abidov's adjuvant | | | | | |
| SHIGELLVAK | 20 | 60 | (36-81)* | 64 | (8-64)* |
| Not administered | 20 | 0 | (0-17) | 0 | (0-0) |

(control)

*differences with control are considered to be significant at p < 0.05;
**differences with SHIGELLVAK vaccinated animals are considered to be significant at p < 0.05.

Immunization with typhoid vaccine. The results shown in Table 5 showed that immunization with the typhoid vaccine “VIANVAK” ensured the formation of stable antibody genesis to Salmonella antigens (in particular, to the S. Typhi O-antigen) in 80% of the immunized animals. At the same time, the titer of specific antibodies in blood serum (Me) was 1:64. The differences with the control were statistically significant (p<0.05) in both cases. The percentage of positive seroconversions reached 100% when using combination of the typhoid vaccine “VIANVAK” with Abidov's adjuvant, which is 20% higher than among those vaccinated with only IMP, and the level of specific serum antibodies (Me) reached 1:256, which significantly exceeded both control values, and a similar indicator in animals vaccinated with only IMP (p<0.05).

Immunization with shigellosis vaccine. The results shown in Table 5 showed that immunization with the vaccine “Shigellvak” ensured immune response formation against Shigella sonnei in 60% of immunized animals, that is, the level of positive seroconversions was 60%, which significantly exceeded the control values (p<0.05). At the same time, the average titer value of specific antibodies in the blood serum of vaccinated mice (Me) was 1:64, which also significantly exceeded the control values (p<0.05). The rate of positive seroconversion reached 100% when using combination of Shigellvak immunization and Abidov's adjuvant, which significantly exceeded both the control values and the same indicator in animals vaccinated with only Shigellvak. There was also a more intensive formation of specific serum antibodies under these conditions, as evidenced by the value of the average titer (Me) equaled 1:256, which was significantly higher by 4 times than in control and in animals vaccinated with only Shigellvak (p<0.05).

### Example 6. Adjuvant Activity of Abidov's Adjuvant in Relation to IMP Against Venezuelan Equine Encephalomyelitis and Orthopoxvirus Infection, Applied as part of the Association (VVE+SLDV)

Dangerous infectious diseases (DID) represent a rather serious epidemiological group of infections, the pathogens of which are characterized by pronounced pathogenicity, virulence and contagiousness in relation to the human population, which contributes to their sufficiently rapid spread, coverage of large contingents and causing serious damage to public health and national security of states on the territories of which they are registered. An example is the recurring epidemics of Ebola, Zika, dengue, West Nile, coronavirus infections, etc. Therefore, close attention of epidemiologists and infectious disease specialists has been and continues to be paid to the issues of prevention and treatment of AIDs, and priority being given to immunological prophylaxis as the most effective measure in relation to this group of infections. Moreover, the complexity in the IMP use is of high priority, since there is a high probability of the occurrence and rapid progression of not one, but several infections at the same time as a result of a sharp deterioration in the sanitary and epidemic situation in a particular region. Recently, the attention of researchers has been riveted to the so-called “complex vaccinal systems” (CVS), which are designed to be an alternative to the existing means of immunological prophylaxis (IP). Such systems include IMP associations and complexes that facilitate the simultaneous administration of several antigenic stimulating agents into the body, as well as IMP on the basis of nanoparticles coated with several antigenic determinants against several infections.

CVS are structurally represented by nanoparticles of different nature, loaded with the most immunologically active determinants of infectious agents [53-55]. The main principle of CVS design is increasing the protection of immunogenic substances from the effects of the body enzyme systems and, as a result, their stability and bioavailability in relation to immunocompetent cells [56].

Considering the above and for evidence completeness of the adjuvant properties of Abidov's adjuvant, we conducted studies to evaluate its adjuvant properties under the conditions of simultaneous/separate administration with the IMP association against VVE and orthopoxvirus infection.

Cultural inactivated liquid VEL was used for immunological prophylaxis of VEL (ser. 145; control No. 1244). Animals were immunized once subcutaneously, the immunizing dose of the drug was 0.1 of human dose. The smallpox live dry vaccine (ser. T. 15, control No. 1542) was used for the immunological prophylaxis of orthopoxvirus infection. The drug was used once subcutaneously in a volume of 0.5 ml at a vaccination dose equivalent to 0.2 of human dose.

Infectious agents:
VVE virus—Trinidad pathogenic strain. The accumulation of virus-containing material for subsequent infection of laboratory animals was carried out using 9-11 day old developing chicken embryos—30-50 pcs. Five consecutive tenfold dilutions of the virus-containing suspension were initially prepared. 0.2 ml of each dilution of the virus-containing suspension was introduced into the allantoic cavity of developing chicken embryos. The administration site of the virus-containing suspension was covered with molten paraffin. Then developing chicken embryos were placed in a thermostat at a temperature of (37±0.5)° C. for 18 hours, periodically assessing their viability using an ovoscope. The viability of developing chicken embryos was assessed after the incubation in thermostat, and a 10% suspension of virus-containing material was prepared from the “carcasses” of live embryos using a physiological solution with the addition of antibiotics (penicillin at the rate of 100 IU per 1 ml, streptomycin — 200 IU per 1 ml). The resulting suspension was centrifuged for 10 min at 1.5-2.0 thousand rpm and a temperature of plus (3±0.5)° C. The supernatant was poured into 1.0 ml vials and used for further infection of experimental animals — mice. The initial virus titer was 10⁷-10⁸ LD₅₀/ml.
CP virus—pathogenic strain Pumenok. The accumulation of virus-containing material for infection of laboratory animals was carried out using 11-12 day old developing chicken embryos—30-50 pcs. Five consecutive tenfold dilutions of the virus-containing material were initially prepared. 0.2 ml of each dilution was applied to the chorion-allantoic membrane of developing chicken embryos. The administration site was covered with molten paraffin. Infected developing chicken embryos were placed in a thermostat at a temperature of (35±0.5)° C. for 72 hours, daily assessing their viability using an ovoscope. The viability of developing chicken embryos was assessed after the incubation in thermostat, and 60 ml of 10% suspension of virus-containing material was prepared from the chorion-allantoic membrane of live embryos using a physiological solution with the addition of antibiotics (penicillin at the rate of 100 IU per 1 ml, streptomycin—200 IU per 1 ml). The resulting suspension of virus-containing material was centrifuged for 10 min at 1.5-2.0 thousand rpm and a temperature of (3±0.5)° C. The supernatant was poured into 10 ml vials (penicillin vials) and used for further infection of laboratory animals. The initial virus titer was 10³-10⁴ LD₅₀/ml.

The studies were performed on white outbred male mice weighing 16-18 g (350 heads) and 10-12 g (180 heads). The experiments were carried out on animals that were obligatorily quarantined for 1 week.

In vivo VVE modeling was carried out on outbred mice weighing 16.0-18.0 g by intranasal infection with virus-containing material in a volume of 0.3 ml. Two infectious doses of the pathogen were used, causing the death of 57-100% of infected animals in the control. The mice were monitored after infection daily for 21 days with registration of the number of living and dead individuals. The incubation period of the simulated infection averaged 5 days. The disease was characterized by the following signs: experimental animals became slow-moving, refused to eat and drink, their hair was disheveled. The phenomena of encephalitis (impaired coordination, the appearance of paresis and paralysis) developed and increased, and the death of infected animals occurred subsequently. The maximum death of mice was noted, as a rule, on the 7-9th day after infection.

OK modeling was carried out on white outbred mice weighing 10.0-12.0 g, after they were subjected to ether Rausch anesthesia, and then intranasal infection with the CP virus was performed in a volume of 0.03 ml in each nostril. Two infectious doses of the pathogen were used in the experiment, which differed by one magnitude. Infected animals were monitored after infection daily for 14 days with registration of the number of living and dead individuals.

Antibodies to the smallpox antigens were determined in the blood serum of animals immunized with the corresponding IMP on the 21st days after immunization. The presence of specific smallpox antibodies in blood sera was determined using conventional serological tests—neutralization reaction (NR), treponema pallidum hemagglutination assay (TPHA), hemagglutination inhibition test (HIT), indirect hemagglutination test (IHT). Antibodies to the antigens of the VVE virus were determined in the blood serum of animals immunized with the corresponding IMP on the 21st days after immunization. The presence of specific antibodies to the VVE virus was determined using generally accepted serological tests—NR, TPHA, HIT, IHT.

Animals were immunized in the course of the studies according to the scheme: animals were initially immunized with the IMP combination (VVE+SLDV), and the adjuvant was administered simultaneously with the IMP combination and then 3 and 6 days after immunization—with the IMP combination intraperitoneally in a volume of 0.5 ml at a dose of 150 μg/individual. Blood was taken from the animals by decapitation before immunization and 21 days after the last administration of Abidov's adjuvant, and the titers of the corresponding specific antibodies were determined in the blood serum. Summary of the results obtained is shown in the Table 6. As follows from the data presented in Table 6, VVE provided 70% of seroconversions, which significantly exceeded the control parameters, since no specific antibodies to the VVE virus were detected in the blood serum of animals in the control group.

TABLE 6
Assessment of the adjuvant activity of Abidov's adjuvant in relation to IMP
against orthopoxvirus infection and Venezuelan equine encephalomyelitis with their
simultaneous/separate use

| | | The values of VVE immunological efficiency indicators when it is | | | | | |
|---|---|---|---|---|---|---|---|
| | | determined by serological methods | | | | | |
| | | | | Treponema pallidum | | Hemagglutination | |
| | | | | hemagglutination | | inhibition | |
| | | Neutralization | | assay | | test | |
| | | reaction | | | Reciprocal | | Reciprocal |
| | Number of | | Reciprocal | | value | | value |
| | immunized | | value of | Sero- | of | Sero- | of |
| Immunization | animals, | Sero- | antibody | conversions, | antibody | conversions, | antibody |
| IMP | heads | conversions, % | titer, Me | % | titer, Me | % | titer, Me |

VVE immunogenicity

| | | | | | | | |
|---|---|---|---|---|---|---|---|
| (VVE + | 20 | 100* | 25 | 100 | 160*)** | 100 | 80 |
| SLDV) + | | (83-100) | (25-125) | (83-100)* | (80-320) | (83-100)* | (40-160) |
| Abidov's | | | | | | | |
| adjuvant | | | | | | | |
| VVE | 20 | 70* | 25 | 70 | 40 | 70 | 80 |
| | | (46-88) | (25-125) | (46-88)* | (40-80) | (46-88)* | (40-80) |
| Not | 20 | 0 | 0 | 0 | 0 | 0 | 0 |
| administered | | (0-17) | (0-0) | (0-17) | (0-0) | (0-17) | (0-0) |
| (control) | | | | | | | |

SLDV immunogenicity

| | | | | | | | |
|---|---|---|---|---|---|---|---|
| (VVE + | 20 | 100* | 25 | 100 | 160*)** | 100 | 80*)** |
| SLDV) + | | (83-100) | (25-125) | (83-100)* | (80-160) | (83-100)* | (80-160) |
| Abidov's | | | | | | | |
| adjuvant | | | | | | | |
| SLDV | 20 | 100* | 25 | 100* | 40 | 100* | 20 |
| | | (83-100) | (25-125) | (83-100) | (20-40) | (83-100) | (20-80) |
| Not | 20 | 0 | 0 | 0 | 0 | 0 | 0 |
| administered | | (0-17) | (0-0) | (0-17) | (0-0) | (0-17) | (0-0) |
| (control) | | | | | | | |

*differences with control are considered to be significant at p < 0.05;
**differences with SLDV vaccinated animals are considered to be significant at p < 0.05.

The immunogenic properties of the vaccine increased when Abidov's adjuvant was administered to animals against the background of IMP. This was manifested both by an increase in the level of positive seroconversions (by 30% compared with indicators of animals vaccinated only with VVE), and specific serum antibody titers, which was most clearly manifested when titrating immune sera by TPHA (p<0.05). Comparison of the obtained results with the results similar in terms of protective efficiency (Table 2) allows us to conclude that the obtained higher survival rates of animals infected with the VVE virus, previously immunized and treated with Abidov's adjuvant together with the vaccine, may be due to more pronounced antibody genesis under the drug influence.

With regard to immunization with SLDV as part of the VVE+SLDV association, it turned out that the smallpox vaccine provided the formation of smallpox immunity in all 20 immunized animals by the 21st day of the post-vaccination period, both when used alone and in association with VVE. The seroconversion rate was 100%, which significantly differed from the control level. At the same time, titers ranged depending on the method of detecting specific smallpox antibodies in the blood serum from 1:25 (in the neutralization reaction) to 1:40 (in the treponema pallidum hemagglutination assay). Since no specific anti-small antibodies were found in the blood serum of the control animals, the differences in titers between SLDV immunized and control mice were considered to be significant at p<0.05. These data are consistent with studies on the assessment of the intensity of post-vaccination immunity, conducted on the CP model caused by the virus of the same name, strain Pumenok. At the same time, there was also no tangible contribution of the drug to the increase in IMP immunogenicity, however, there was no negative effect of Abidov's adjuvant on the immunological efficiency of SLDV. It cannot be denied, however, that the processes of anti-smallpox antibody genesis were intensified under its influence, which manifested itself in an increase in the titer of specific anti-smallpox antibodies in the blood serum of animals immunized with the combination of drugs by 4 times compared with same indicators in animals vaccinated with only SLDV, and the differences were statistically significant (p<0.05).

Summarizing the results of the conducted studies, it can be concluded that:
Abidov's adjuvant is a promising immunotropic drug with adjuvant activity in relation to IMP of various nature against infectious agents that cause dangerous and especially dangerous infections of a bacterial, viral and toxin nature;
Abidov's adjuvant shows adjuvant properties when used at a dose of 150 μg/individual, administered once, intramuscularly both simultaneously/separately from IMP, and against the background of ongoing immunization process, either 3-1 days before the expected infection, or simultaneously with IMP and then 3 days, 6 days after the IMP introduction;
adjuvant properties of Abidov's adjuvant consist in stimulation of specific antibody genesis by at least 30% compared to that under the influence of sole IMP, and this effect is most pronounced when using the adjuvant in combination with inactivated and chemical vaccines, as well as polyanatoxins;
Abidov's adjuvant in combination with live vaccines, using the example of SLDV, does not lead to an increase in the immunogenic properties of such drugs, however, it also does not have a negative immunotropic effect. Considering the anti-inflammatory effect of Abidov's adjuvant, its use with live vaccines is justified in terms of reducing their reactogenicity due to the pro-inflammatory effects of such IMPs at the beginning of the post-vaccination period, especially at the stage of the primary IgM response;
Abidov's adjuvant has a positive immunotropic effect in relation to not only monovaccines, but also vaccine preparations or immunoantigens in the composition of associated drugs (TA) and complex vaccine systems (VVE+SLDV association). Moreover, an increase in immunogenicity occurs almost to the same extent with respect to each of the antigens included in the association or complex, with the exception of live vaccines. With regard to the latter, the use of the adjuvant is not so much aimed at increasing their immunogenicity, but rather at reducing the vaccination reactogenicity and, as a result, increasing the safety of such immunization;
adjuvant action mechanism of Abidov's adjuvant is due to the simultaneous launch under its influence of non-specific immunological resistance systems, cytokines and chemokines, an increase in the intensity of processing of antigens to immunogenic forms and, as a result, more pronounced stimulation of specific antibody genesis. Moreover, Abidov's adjuvant has the ability not only to act as an immunological adjuvant, but also, possibly, prevents the development of the pathological process of infections at the level of the immune system when entering the body together with IMP. In particular, the latter is known to be associated with immune dysfunctions that develop under the influence of either the infectious agents or their metabolic products. At the same time, there is an imbalance in the functioning of both immunocompetent cells and cells of the nonspecific immunological resistance system, as well as a violation of their secretory activity, which ultimately leads to an imbalance in the mediator component of the immune system;
taking into account the results obtained, proving that Abidov's adjuvant belongs to immunological adjuvants that exhibit a similar effect in relation to IMP of various nature and direction, the following priority areas of its application in immunology and vaccinology are assumed:
   as part of schemes for the immunological prophylaxis of infectious diseases of a bacterial, viral and other nature, including the use of existing IMP or their associations and complexes based on inactivated, chemical recombinant drugs due to insufficient immunogenicity of such drugs;
   as part of immunobiological drugs on the basis of weakly immunogenic antigens (antigenic determinants, protective antigens, components of the outer membranes of bacteria and viruses responsible for their pathogenic and virulent properties, etc.) in the capacity of the IMP structure components;
   as a drug that increases safety without reducing the immunogenicity of the drug in schemes for the immunological prophylaxis of infections of various nature using live vaccines (for immunization against plague, tularemia, anthrax, brucellosis, yellow fever, etc.);
   as a substitute for traditional sorbent substances (aluminium hydrate, etc.) in the production of sorbed mono- or polyanatoxins, inactivated vaccines, etc.

The results obtained should be considered as a rationale for the use of Abidov's adjuvant in both adults and children when conducting immunological prophylaxis of infectious diseases of various nature, including herpes simplex, Venezuelan equine encephalomyelitis, orthopoxvirus infections, botulism, tetanus, typhoid fever, Sonne's dysentery, etc.

### REFERENCES

1. [symbol] [symbol] [symbol] / [symbol] [symbol] [symbol] [symbol] ].-M.: [symbol] [symbol] 2005.-511 c.;
2. Den biologiske trussel og det biologiske beredskab i Danmark/E. D. Heegaard [et al.]//Ugeskr. Laeger.-2005.-Vol. 167, [symbol] 236.-P. 3381-3384.
3. [symbol] A. C. [symbol] [symbol] [symbol] [symbol] /A. C. [symbol] // [symbol] -1998.- [symbol] 5.-C. 5-11.
4. CBR Threats: A case study/K. Cuneo [et al.]//Crisis Res. J.-2005.-Vol. 1, [symbol] 3.-P. 50-52.;
5. Public Health Assessment of Potential Biological Terrorism Agents/L. D. Ron [et al.]//Emerging Infectious Diseases.-2002.-Vol. 8, [symbol] 2.-P. 18-24.
6. VienPoint: Terrorism and dispelling the myth of a panic prone public/B. Sheppard [et al.]//J. Public Health Policy.-2006.-Vol. 27, [symbol] 3.-P. 219-245
7. [symbol] [symbol] [symbol] /Γ. FΓ [symbol] // [symbol] Poc. [symbol] Hayκ.-2003.-T. 73, [symbol] 3.-C. 194-204.
8. Medzhitov R, Schneider D S, Soares M P. Disease tolerance as a defense strategy. Science. 2012; 335(6071):936-941.
9. Ashida H, Mimuro H, Ogawa M, et al. Host-pathogen interactions: cell death and infection: a double-edged sword for host and pathogen survival. J. Cell. Biol. 2011 ; 195(6): 931-942.
10. Tones B Y, Oliveira J H M, Thomas Tate A, Rath P, Cumnock K, Schneider D S. Tracking resilience to infections by mapping disease space. PLoS Biol. 2016;14(4): e1002436-19.
11. Maeurer M, Rao M, Zumla A. Host-directed therapies for antimicrobial resistant respiratory tract infections. Curr. Opin. Pulm. Med. 2016; 22(3):203-211.
12. Monticelli L A, Sonnenberg G F, Abt M C, et al. Innate lymphoid cells promote lung-tissue homeostasis after infection with influenza virus. Nature. 2011; 12(11):1045-1054.
13. Jamieson A M, Pasman L, Yu S, et al. Role of tissue protection in lethal respiratory viral-bacterial coinfection. Science. 2013 ; 340(6137): 1230-1234.
14. Rock J R, Hogan B L M. Epithelial progenitor cells in lung development, maintenance, repair, and disease. Annu. Rev. Cell. Dev. Biol. 2011; 27(1):493-512.
15. [symbol] M. T. [symbol] [symbol] [symbol] [symbol] 2000.
16. [symbol] A. A. [symbol] [symbol] -M.: [symbol] 1999.-607 C.
17. Hackett C. J. Focus on the immunological of effective vaccines//Immunologist -1997.-Vol. 5, [symbol] 5.-P. 171-174.
18. Lee J. J., Sinha K. A., Harrison J. A., et al. Tetanus toxin fragment C expressed in live Salmonella vaccines enhances antibody responses to its fusion partner Schistosoma haematobium glutathione S-transferase//Infect. Immun.-2000.-Vol. 68, [symbol] 5.-P. 2503-2512.
19. [symbol] P. B., [symbol] P. M. [symbol] [symbol] [symbol] -M.: [symbol] 1988.-288 c.
20. [symbol] H. B. [symbol] -M: [symbol] 1999.-279 c.
21. Ertl H. C. J., Xiang Z. Novel vaccine approaches//J. Immunol.-1996.-Vol. 156.-P. 3579-3582.
22. Pardoll D. M. Paracrine cytokine adjuvants in cancer immunotherapy//Ann. Rev. Immunol.-1995.-Vol. 13.-P. 399-415.
23. Kim J. J., Yang J. S., Dentchev T., Dang K., Weiner D. B. Chemokine gene adjuvants can modulate immune responses induced by DNA vaccines//J. Interferon Cytokine Res.-2000.-Vol. 20, [symbol] 5.-P. 487-498.
24. [symbol] [symbol] [symbol] [symbol] [symbol] [symbol] [symbol] [symbol] [symbol] [symbol] [symbol] //Intern. J. Immunorehabilitation.-1997.- [symbol] 6.-C. 35-40.
25. Pasquini S., Xiang Z., Wang Y., et al. Cytokines and cjstimulatory molecules as genetic adjuvants//Immunol. Cell Biol.-1997.-Vol. 75. [symbol] 4.-P. 397-401.
26. Fleo J., Tisminetzky S., Baralle F. Modulation of the immune response to DNA vaccine by costimulatory molecules//Immunology.-2000.-Vol. 100, [symbol] 2.-P. 259-267.
27. Kim J. J., Yang J. S., Montaner L., Lee D. J., Chalian A. A., Weiner D. B. Coimmunization with IFN-gamma or IL-2, but not IL-13, or IL-4 cDNA can enhance Th-1 type DNA vaccine-induced immune responses in vivo//J. Interferon Cytokine Res.-2000.-Vol. 20, [symbol] 3,-P. 311-319.
28. Iwasaki A., Stiernholm B. J. N., Chan A. K., Berinstrein N. L., and Barber B. H. Enhanced CTL responses mediated by plasmid DNA immunogens encoing costimulatory molecules and cytokines//J. Immunol.-1997.-Vol. 158.-P. 4591.
29. Tough D. F., Sun S., Zhang X., Sprent J. Stimulation of naive and memory T cells by cytokines//Immunol. Rev.-1999.-Vol. 170.-P. 39-47.
30. [symbol] P. B., [symbol] P. M. [symbol] [symbol] [symbol] [symbol] : [symbol] [symbol] [symbol] [symbol] [symbol] //Intern. J. Immunorehabilitation.-1999.- [symbol] 11.-C 13-25.
31. Mebra N. K. Major histocompatibility complex and future vaccination strategies//Proc. Indian. Nat. Sci. Acad. B.-1998.-Vol. 64.- [symbol] 2.-P. 81-100.
32. Lindblad E. B., Elhay M. J., Silva R., Appelberg R., Andersen P. Adjuvant modulation of immune responses to tuberculosis subunit vaccines//Infect. Immun.-1997.-Vol. 65, [symbol] 2-P. 623-629.
33. Sjolander A., Bengtsson K. L., Morein B. Kinetics, localisation and cytokine profile of T-cell responses to immune stimulating complexes (iscoms) containing human influenza virus envelope glycoproteins//Vaccine.-1997.-Vol. 15, [symbol] 9.-P. 1030-1038.
34. Gursel M., Gregoriadis G. The immunological co-adjuvant action of liposomal interleukin-2: the role of mode of localisation of the cytokine and antigen in the vesicles//J. Drug Target.-1998.-Vol. 5, [symbol] 2.-P. 93-98.
35. Krup O. C., Kroll I., Bose G., Falkenberg F. W. Cytokine depot formulations as adjuvants for tumor vaccines. I. Liposome-encapsulated IL-2 as a depot formulation//J. Immunother.-1999.-Vol. 22, [symbol] 6.-P. 525.
36. Katz J. M., Lu X., Young S. A., Galphin J. C. Adjuvant activity of the heat-labile enterotoxin from enterotoxigenic Escherichia coli for oral administration of inactivated influenza virus vaccine//J. Infect. Dis.-1997.-Vol. 175, [symbol] 2.-P. 352-363.
37. Rajananthanan P., Attard G. S., Sheikh N. A., Morrow W. J. Novel aggregate structure adjuvants modulate lymphocyte proliferation and Th1 and Th2 cytokine profiles in ovalbumin immunized mice//Vaccine.-1999.-Vol. 18, [symbol] 1-2.-P. 140-152.
38. [symbol] C. A., [symbol] A. C., [symbol] A. A. [symbol] [symbol] - [symbol] 1992.-C. 256 c.
39. [symbol] C, A., [symbol] H. M. [symbol] [symbol] - [symbol] : [symbol] 1998.-156 c.
40. [symbol] [symbol] [symbol] - [symbol] : [symbol] 1998.-112 c.
41. Pighetti G. M., Sordillo L. M. Enhanced antigen-specific responses in bovine mammary glands following administration of interleukin-2//J. Dairy Sci.-1995.-Vol. 78, N 23.-P. 528-537.
42. Souberbielle B. E., Knight B. C., Morrow W. J., et al. Comparison of IL-2- and IL-4-transfected B16-F10 cells with a novel oil-microemulsion adjuvant for B16-F10 whole cell tumour vaccine//Gene Ther.-1996.-Vol., [symbol] 10.-P. 853-858.
43. [symbol] A. B. [symbol] [symbol] [symbol] [symbol] [symbol] [symbol] // [symbol] .-2010.- [symbol] 2.-C15-18.
44. [symbol] [symbol] [symbol] P. [symbol] [symbol] T. 6, M., 1960, c. 160.
45. [symbol] [symbol] P. A., [symbol] [symbol] T. 8, M., 1985, c. 160-84.
46. RU2492527 dated Oct. 9, 2013.
47. RU2345774 dated Oct. 2, 2009.
48. RU2332998 dated Oct. 9, 2008.
49. RU56185 dated Oct. 9, 2006.
50. RU2011107380 dated 27 Aug. 2012.
51. [symbol] [symbol] A. A. [symbol] [symbol] [symbol] [symbol] - [symbol] [symbol] 1962.-178 c.
52. [symbol] B. C. [symbol] [symbol] [symbol] [symbol] [symbol] [symbol] -M.: Hayica, 1967.-208 C.
53. Fan, Y. Cationic liposome-hyaluronic acid hybrid nanoparticles for intranasal vaccination with subunit antigens/Y. Fan [et al.]//J. Control Rel.-2015.-Vol. 208.-P. 121-129.
54. Haughney, S. L. Effect of nanovaccine chemistry on humoral immune response kinetics and maturation/S. L. Haughney [et al.]//Nanoscale.-2014.-Vol. 6.-P. 13770-13778
55. Irvine D. J. Synthetic Nanoparticles for Vaccines and Immunotherapy/D. J. Irvine [et al.]//Chem. Rev.-2015.-Vol. 115.-P. 11109-11146.
56. Sahdev, P. Biomaterials for nanoparticle vaccine delivery systems/P. Sahdey [et al.]//Pharm. Res.-2014.-Vol. 31.-P. 2563-2582.

1. Vaccine for the immunological prophylaxis of infectious diseases, containing a derivative of synthetic derivatives of phthalhydrazide (Abidov's adjuvant) as an adjuvant, as well as a pharmaceutically acceptable carrier and/or diluent.

2. The use of derivative of synthetic phthalhydrazide derivatives (Abidov's adjuvant) as an adjuvant to increase the immunogenicity of agents for specific immunological prophylaxis of infectious diseases, regardless of their nature.

3. Immunogenicity increasing method of agents for specific immunological prophylaxis of infectious diseases, regardless of their nature, including the use of a derivative of synthetic phthalhydrazide derivatives (Abidov's adjuvant):
by intramuscular administration simultaneously with the antigen or immunogen, but in different syringes; or
by intramuscular administration three times: 3 days, 2 days, 1 day before antigen or immunogen administration;
or administration together with antigen or immunogen.

4. The method according to p. 2, where Abidov's adjuvant is used in a single dose of 300 μg/ml (150 μg/individual) intramuscularly in a syringe when administered separately from an antigen or immunogen; used at a dose of 0.01 to 1000 mg/ml simultaneously with the antigen or immunogen introduction, while the immunogenicity of inactivated and chemical vaccines is increased by at least 3 times, the immunogenicity of live vaccines is increased without affecting their safety by means of reducing reactogenicity, polyanatoxin association immunogenicity increases by at least 30% for each of the antigen (anatoxin) included in the association.
